(19) United States
(12) Patent Application Publication
(10) Pub. No.: US 2018/0367876 A1
(43) Pub. Date: Dec. 20, 2018

(54) Salt Rock Speaker System

(71) Applicant: James R. Story, III, Lawrenceburg, TN (US)

(72) Inventor: James R. Story, III, Lawrenceburg, TN (US)

(21) Appl. No.: 15/374,813

(22) Filed: Dec. 9, 2016

(51) Int. Cl. H04R 1/02; F21V 33/00; F21V 23/04; A61K 41/00; B03C 3/34

(52) U.S. Cl. CPC H04R 1/028; F21V 33/0056; F21V 23/04; A61K 41/0004; F21Y 2101/00; H04R 2420/07; H04R 2420/09; H04R 2430/01; B03C 3/34

(57) ABSTRACT

A speaker system to distribute sound may include a housing formed from Himalayan salt rock having a central aperture; a platform being positioned within the central aperture; a light source being mounted on the platform;
a base connected to the housing; and a speaker connected to the base at an acute angle.

### FIELD OF THE INVENTION

The present invention relates to entertainment systems and more particularly to a system for creating a soothing environment.

### BACKGROUND

In today's modern society, there never seems to be enough time to perform all the required functions. As a result, lack of sleep and stress are caused by the efforts of people to finish these required functions.

The present invention releases this stress.

Himalayan salt is rock salt or halite from the Punjab region of Pakistan. It is mined at the Khewra Salt Mine in Khewra, Jhelum District, Punjab and other locations in the Himalayans, which is situated in the foothills of the Salt Range hill system in the Punjab province of the Indo-Gangetic Plain. Himalayan salt has said to provide benefits of air cleaning, medical benefits, ions emitted, positive energy waves and neutralizing electromagnetic radiation.

### SUMMARY

A speaker system to distribute sound may include a housing formed from Himalayan salt rock having a central aperture; a platform being positioned within the central aperture; a light source being mounted on the platform;

a base connected to the housing; and a speaker connected to the base at an acute angle.

The light source may be an incandescent type of bulb;

The light source may be a multi spectrum LED light.

The speaker may be a Bluetooth speaker;

The speaker may be a Wi-Fi speaker.

### BRIEF DESCRIPTION OF THE DRAWINGS

The invention may be understood by reference to the following description taken in conjunction with the accompanying drawings, in which, like reference numerals identify like elements, and in which:

FIG. 1a illustrates an exploded view of the Himalayan salt rock housing system;

FIG. 1b illustrates a bottom view of the Himalayan salt rock housing system;

FIG. 2a illustrates a cross-sectional side view of the Himalayan salt rock housing system;

FIG. 2b illustrates the bottom view of the Himalayan salt rock housing system;

FIG. 3a illustrates a bottom view of the support device of the present invention;

FIG. 3b illustrates a perspective view of the support device of the present invention;

FIG. 3c illustrates a side view of the support device of the present invention;

FIG. 4 illustrates another view of the Himalayan rock salt system of the present invention;

FIG. 5 illustrates a block diagram of the electronics and communications of the Himalayan salt rock system.

FIG. 6a illustrates an exploded view of another embodiment of the Himalayan salt rock housing system;

FIG. 6b illustrates a bottom view of the another embodiment of the Himalayan salt rock housing system;

FIG. 7a illustrates a cross-sectional side view of the another embodiment of the Himalayan salt rock housing system;

FIG. 7b illustrates the bottom view of the another embodiment of the Himalayan salt rock housing system;

FIG. 8a illustrates a bottom view of the another embodiment of the support device of the present invention;

FIG. 8b illustrates a perspective view of the another embodiment of the support device of the present invention;

FIG. 8c illustrates a side view of the another embodiment of the support device of the present invention;

FIG. 9a illustrates a side view of the another embodiment of the Himalayan rock salt system of the present invention;

FIG. 9b illustrates a bottom view of the another embodiment of the Himalayan rock salt system of the present invention;

FIG. 9c illustrates a side view of a piece of the another embodiment of the Himalayan rock salt system of the present invention;

FIG. 10 illustrates a block diagram of the electronics and communications of the Himalayan salt rock system;

FIG. 11 illustrates a view of the another embodiment of the Himalayan salt rock system.

### DETAILED DESCRIPTION

The present invention combines an audio speaker and light bulb into a Himalayan salt rock housing of any shape, providing a warm glowing relaxing light source, not only producing negative ions which helps cleanse the surrounding air but also creates a relaxed atmosphere to release STRESS by listening to your favorite music or sounds of the ocean or forest etc.

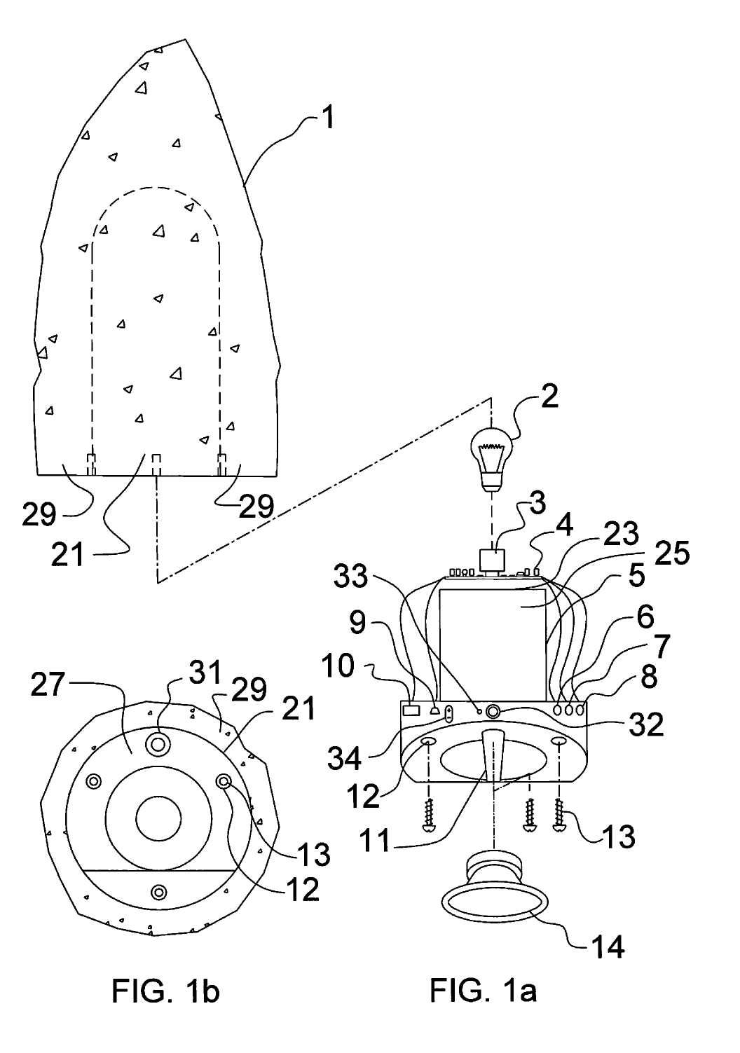

FIG. 1a illustrates the Himalayan salt rock housing 1 and the Bluetooth and or Wi-Fi speaker 14 or other type of speaker and a lighting device 2 which may include a platform 5 and a fastener 13 which may be threaded screws to cooperate with apertures 12 formed into the bottom surface of the Himalayan salt rock housing 1. Other types of fasteners are within the scope of the present invention. The platform 5 may be mounted on a base 27 (the speaker housing) which may connect to the bottom surface 29 of the Himalayan salt rock housing 1 by a multitude of fasteners 13 which may be threaded screws which may extend through the base 27 and engage the bottom surface 29 of the Himalayan salt rock housing 1.

The Himalayan salt rock housing 1 may be any suitable shape and may include a central aperture 21 which may extend upwards from the bottom surface 29 to accommodate the platform 5 which may include a top wall 23 which may be connected to a multitude of sidewalls 25 connected to the top wall 23 to form an inverted U-shaped (in cross-section) platform 5. The housing 1 may be solid

The top wall 23 may be connected to a socket 3 to connect to a light source 2 which may be a standard incandescent type low voltage bulb or a multi spectrum light emitting diode LED that may or may not be Philips HUE compatible. The light source 2 be switchable on/off by switch 4 and may be controllable by both hardware and software. If the light source 2 is the light-emitting diode LED, the lighting may be If This Then That (Internet-based services) IFTTT compatibility, a beat detect feature to detect the beat of the sounds, brightness control for the light source, Image Output Terminal IOT compatible, voice message command sensitive to allow the user control, preprogrammed light shows, and thru software the ability for users to create their own light show, hue control via hardware and software.

The speaker 14 may be mounted in the base 27 (speaker housing) having a driver facing down and in one embodiment the driver may be positioned at a 22-33 degree angle or alternatively at a 0-135° angle to maximize audio quality dependent on the driver and soundscape desired.

FIG. 1a additionally illustrates a USB port 10 connected to the base 27.

Furthermore, the switch 6 which may be a toggle switch controls the source of the sound for the speaker 144 example the source of sound may be the sounds of nature, and a power indicator 7 provides an indication that power is being delivered to the Himalayan salt rock system 100. A power switch 8 controls the power supplied to the Himalayan salt box system 100 for example turning the power on and off; a first USB port 9 (female port) allows for various types of electronic connections; a second USB port 10 (female port) allows for remote charging. FIG. 1a additionally illustrates far field microphone 33; a volume control 34 to control the volume of the speaker 14, an audio jack 35 which may be a 3.5 mm audio jack or other size to allow the output of audio from the Himalayan salt rock system 100. In addition, FIG. 1a illustrates a camera 32 to record video.

FIG. 1b illustrates a bottom view of the base 27, the fasteners 13 and the aperture 12 through the bottom surface 29 of the Himalayan salt rock housing 1. The base may include a support leg 31 to support the base 27 and the Himalayan salt rock housing 1.

Himalayan salt rock system 100 provides benefits of air cleaning, medical benefits, ions emitted, positive energy waves and neutralizing electromagnetic radiation.

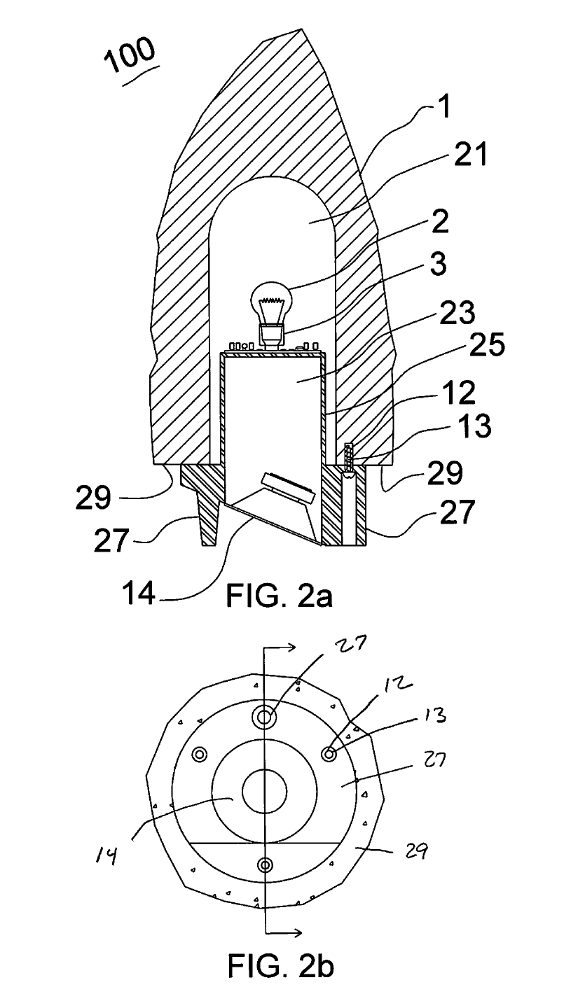

FIG. 2a illustrates a cross-sectional view of the Himalayan salt rock system 100 of the present invention and illustrates the Himalayan salt rock housing 1 having a central aperture 21 being defined by bottom surface 29.

Additionally illustrated is the platform 5 (the speaker housing) which connects to the socket 3 to connect to the light source 2 and which is connected to the base 27 for mounting the speaker 14 which is connected to the bottom surface 29 of the Himalayan salt rock housing 1. The fasteners 13 cooperate with the aperture 12 to hold the platform 5 and the base 27 in position with respect to the housing 1.

FIG. 2b illustrates the bottom surface 29 of the Himalayan salt rock housing 1 which may be connected to the base 27 by fasteners 13 and apertures 12. The base 27 may be connected to the speaker 14.

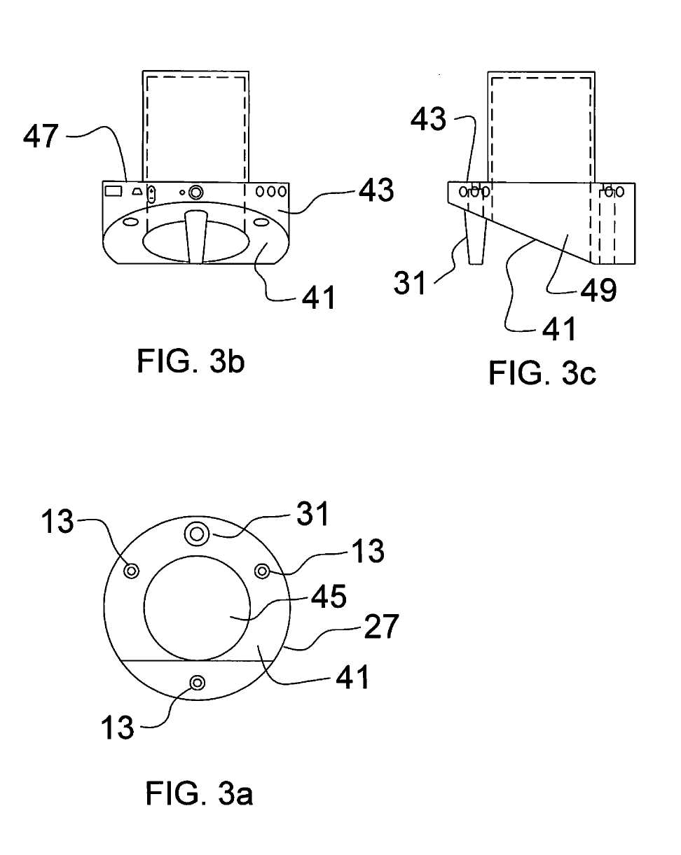

FIG. 3a shows a bottom view of the base 27 without the speaker 14 and illustrates the base bottom surface 41, the fasteners 13 and the support leg 31.

FIG. 3b is a perspective view of the base 27 without the speaker 14 and illustrates the support leg 31 extending from the base bottom surface 41 which may be connected to the side surface 43 which may extend around the base aperture 45 which may extend through the base 27. The side surface 43 may be connected to the top surface 47 and the side surface 43 may include an inclined portion 49 (see FIG. 3c) to cooperate with the speaker 14 mounted at an acute angle with respect to the support surface (not shown).

FIG. 3c illustrates a side view of the support device of the present invention.

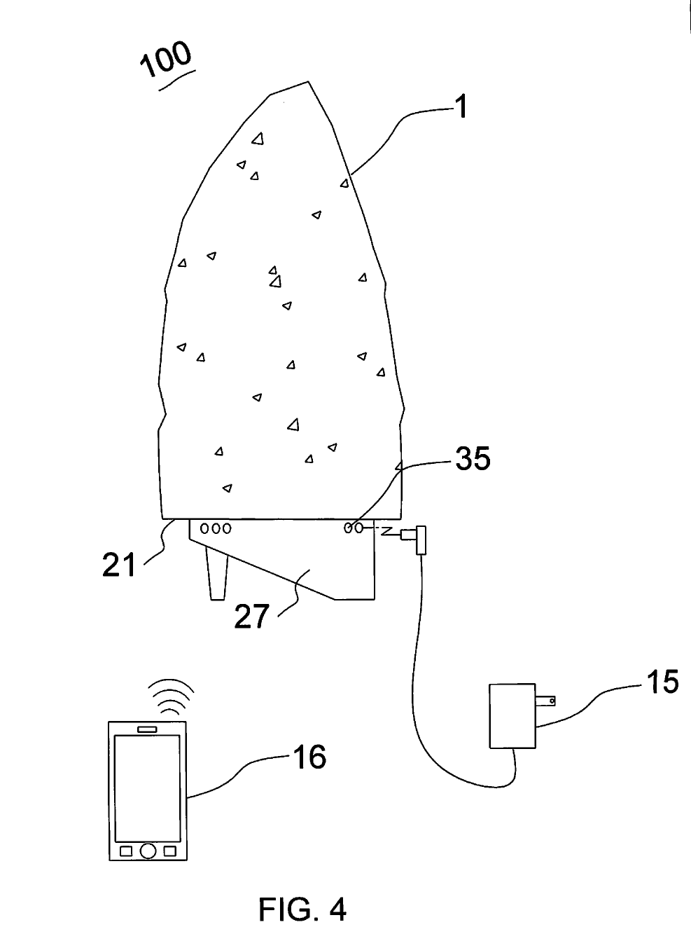

FIG. 4 illustrates the Himalayan salt rock system 100 being powered by a power supply 15 which may include a transformer to reduce the voltage supplied to the base 27 and illustrates a remote controller 16 controlling the Himalayan salt rock system 100 wirelessly and remotely.

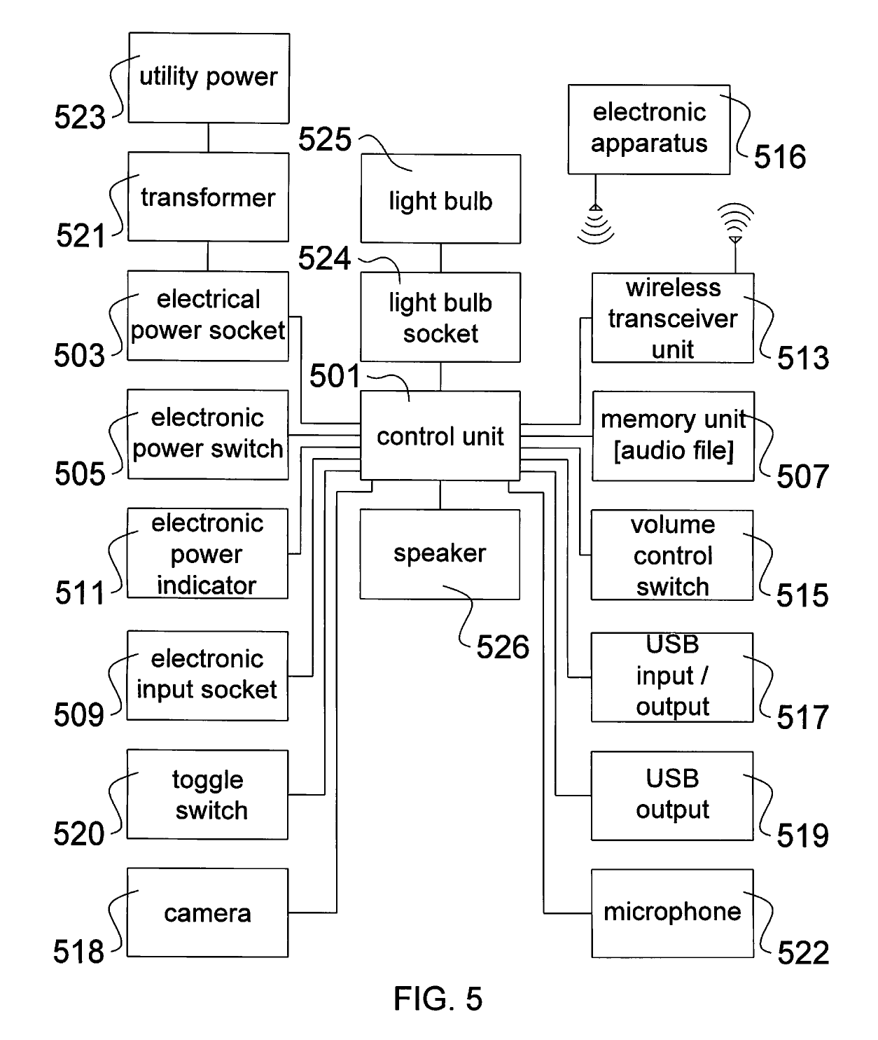

FIG. 5 illustrates a block diagram of the present invention and illustrates a control unit 501 being connected to an electrical power socket 503 to supply power, being connected to an electronic power switch 505 to control the on/off functions, being connected to a memory unit 507 which may store audio files for playback, being connected to electronic input socket 509, and being connected to an electronic power indicator 511 to provide a visual indication of the power.

The control unit 501 may be connected to the wireless transceiver unit 513 which receives signals from the electronic apparatus 515, may be connected to the volume control switch 515 to control the volume of the speaker 14, may be connected to the electronic output socket 517 for control of an electronic device (not shown) and may be connected to another electronic output socket 519 for control of another electronic device (not shown). A transformer 521 may be connected to the electrical power socket 503 and may be connected to the utility power 523.

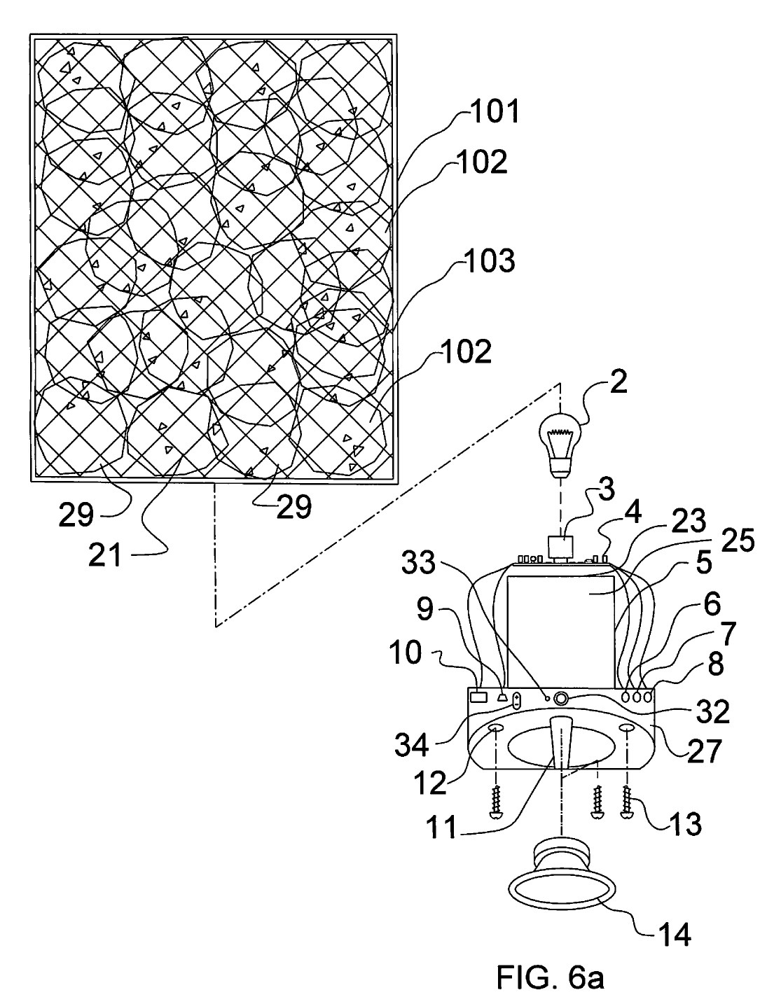

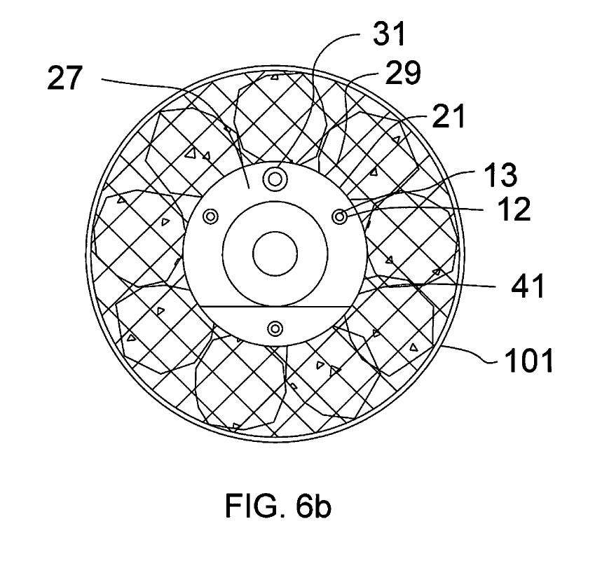

FIG. 6a illustrates an exploded view of another embodiment of the Himalayan salt rock housing system.

FIG. 6a illustrates the Himalayan salt rock housing 101 which may be formed from a multitude Himalayan salt rock pieces 103 of varying size and shape being uniform or not uniform (individual rocks) and a container 103 which may be a lattice of wire 102 (the container may be totally formed of the lattice of wire or partially formed of the lattice of wire) formed from metal, plastic or other appropriate material.

The Himalayan salt rock housing 101 may include the Bluetooth and or Wi-Fi speaker 14 or other type of speaker and a lighting device 2 which may include a platform 5 and a fastener 13 which may be threaded screws to cooperate with apertures 12 formed into the bottom surface of the Himalayan salt rock housing 101 or the fasteners may be hooks to engage the wire 102 of the lattice having a threaded end for a bolt. Other types of fasteners are within the scope of the present invention. The platform 5 may be mounted on a base 27 (the speaker housing) which may connect to the bottom surface 29 of the Himalayan salt rock housing 101 by a multitude of fasteners 13 which may be threaded screws which may extend through the base 27 and engage the bottom surface 29 of the Himalayan salt rock housing 101.

The Himalayan salt rock housing 101 may be any suitable shape and may include a central aperture 21 which may extend upwards from the bottom surface 29 to accommodate the platform 5 which may include a top wall 23 which may be connected to a multitude of sidewalls 25 connected to the top wall 23 to form an inverted U-shaped (in cross-section) platform 5.

The top wall 23 may be connected to a socket 3 to connect to a light source 2 which may be a standard incandescent type low voltage bulb or a multi spectrum light emitting diode LED that may or may not be Philips HUE compatible. The light source 2 be switchable on/off by switch 4 and may be controllable by both hardware and software. If the light source 2 is the light-emitting diode LED, the lighting may be If This Then That (Internet-based services) IFTTT compatibility, a beat detect feature to detect the beat of the sounds, brightness control for the light source, Image Output Terminal IOT compatible, voice message command sensitive to allow the user control, preprogrammed light shows, and thru software the ability for users to create their own light show, hue control via hardware and software.

The speaker 14 may be mounted in the base 27 (speaker housing) having a driver facing down and in one embodiment the driver may be positioned at a 22-33 degree angle or alternatively at a 0-135° angle to maximize audio quality dependent on the driver and soundscape desired.

FIG. 6a additionally illustrates a USB port 10 connected to the base 27.

Furthermore, the switch 6 which may be a toggle switch controls the source of the sound for the speaker 144 example the source of sound may be the sounds of nature, and a power indicator 7 provides an indication that power is being delivered to the Himalayan salt rock system 100. A power switch 8 controls the power supplied to the Himalayan salt box system 100 for example turning the power on and off; a first USB port 9 (female port) allows for various types of electronic connections; a second USB port 10 (female port) allows for remote charging. FIG. 6a additionally illustrates far field microphone 33; a volume control 34 to control the volume of the speaker 14, an audio jack 35 which may be a 3.5 mm audio jack or other size to allow the output of audio from the Himalayan salt rock system 100. In addition, FIG. 6a illustrates a camera 32 to record video.

FIG. 6b illustrates a bottom view of the another embodiment of the Himalayan salt rock housing system 100.

FIG. 6b illustrates a bottom view of the base 27, the fasteners 13 and the aperture 12 through the bottom surface 29 of the Himalayan salt rock housing 101. The base may include a support leg 31 to support the base 27 and the Himalayan salt rock housing 101.

Himalayan salt rock system 100 provides benefits of air cleaning, medical benefits, ions emitted, positive energy waves and neutralizing electromagnetic radiation.

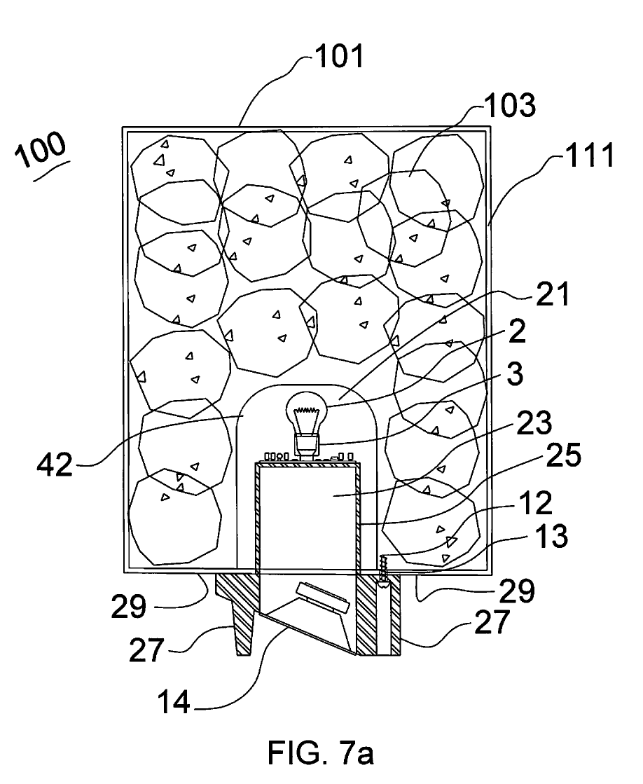

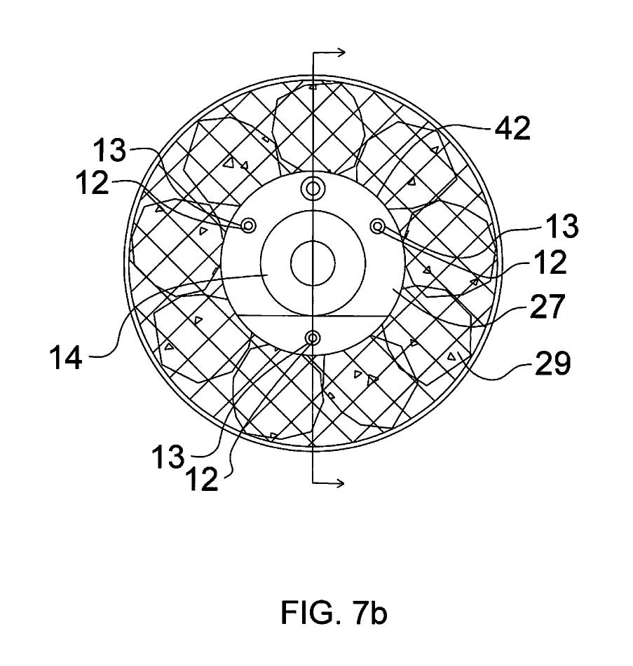

FIG. 7a illustrates a cross-sectional side view of another embodiment of the Himalayan salt rock housing system.

FIG. 7a illustrates a cross-sectional view of the Himalayan salt rock system 100 of the present invention and illustrates the Himalayan salt rock housing 101 having a central aperture 21 being defined by bottom surface 29.

Additionally illustrated is the platform 5 (the speaker housing) which connects to the socket 3 to connect to the light source 2 and which is connected to the base 27 for mounting the speaker 14 which is connected to the bottom surface 29 of the Himalayan salt rock housing 101. The fasteners 13 cooperate with the aperture 12 to hold the platform 5 and the base 27 in position with respect to the housing 101.

FIG. 7b illustrates the bottom view of another embodiment of the Himalayan salt rock housing system.

FIG. 7b illustrates the bottom surface 29 of the Himalayan salt rock housing 101 which may be connected to the base 27 by fasteners 13 and apertures 12. The base 27 may be connected to the speaker 14.

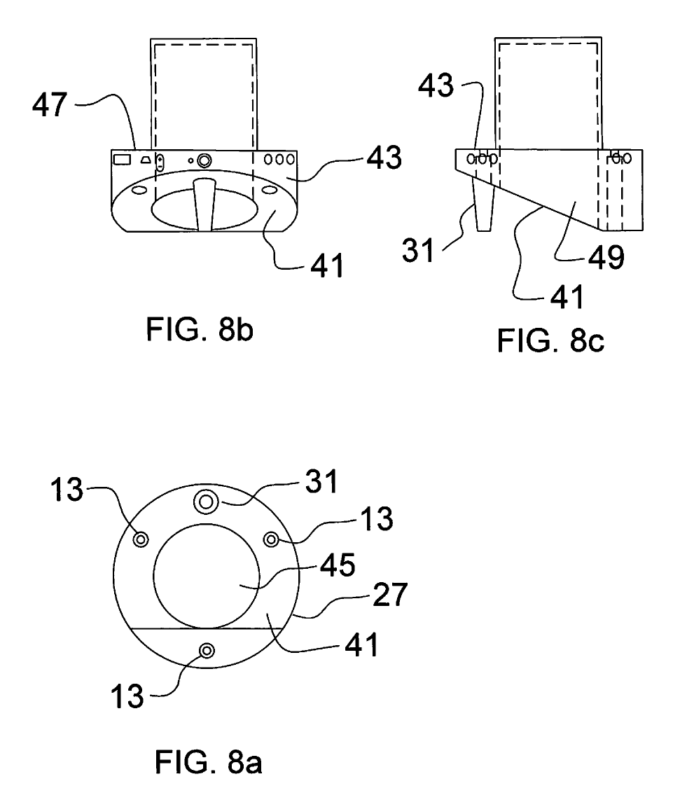

FIG. 8a illustrates a bottom view of another embodiment of the support device of the present invention.

FIG. 8a shows a bottom view of the base 27 without the speaker 14 and illustrates the base bottom surface 41, the fasteners 13 and the support leg 31.

FIG. 8b illustrates a perspective view of the another embodiment of the support device of the present invention, FIG. 8b is a perspective view of the base 27 without the speaker 14 and illustrates the support leg 31 extending from the base bottom surface 41 which may be connected to the side surface 43 which may extend around the base aperture 45 which may extend through the base 27. The side surface 43 may be connected to the top surface 47 and the side surface 43 may include an inclined portion 49 (see FIG. 8c) to cooperate with the speaker 14 mounted at an acute angle with respect to the support surface (not shown).

FIG. 8c illustrates a side view of the other embodiment of the support device of the present invention.

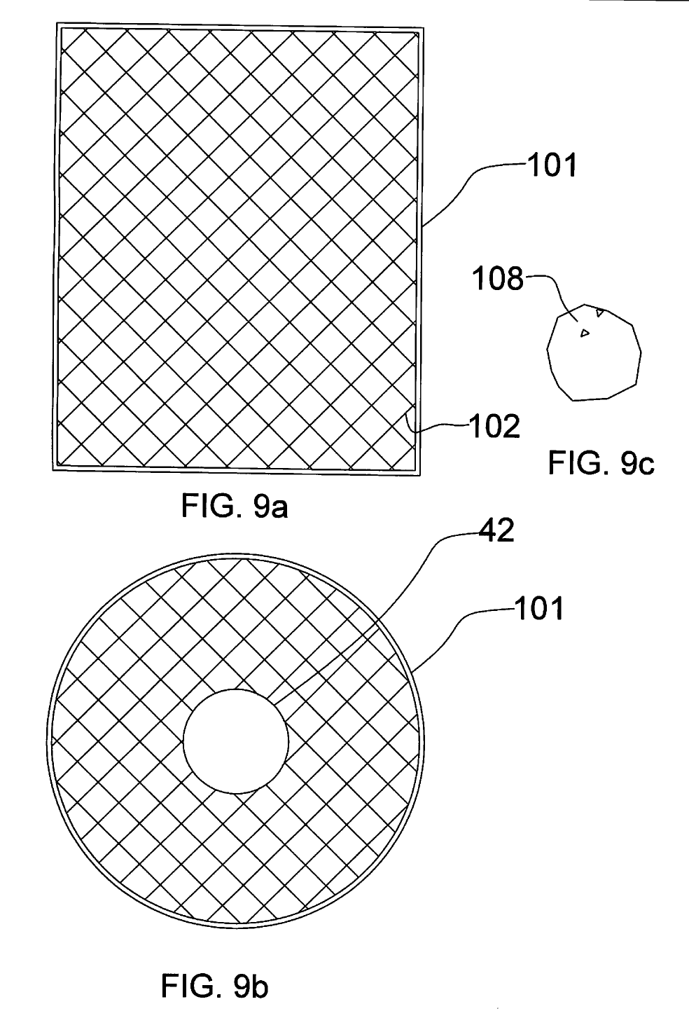

FIG. 9a illustrates another view of another embodiment of the Himalayan rock salt system 100 of the present invention.

FIG. 9a illustrates the Himalayan salt rock housing 101 which may be formed from a multitude of Himalayan salt rock pieces 102 (individual rocks, not shown) and a container which may be a lattice of wire 103 formed from metal, plastic or other appropriate material.

FIG. 9b illustrates another view of the another embodiment of the Himalayan rock salt system of the present invention;

FIG. 9c illustrates a view of the piece 103 of Himalayan rock salt of the present invention.

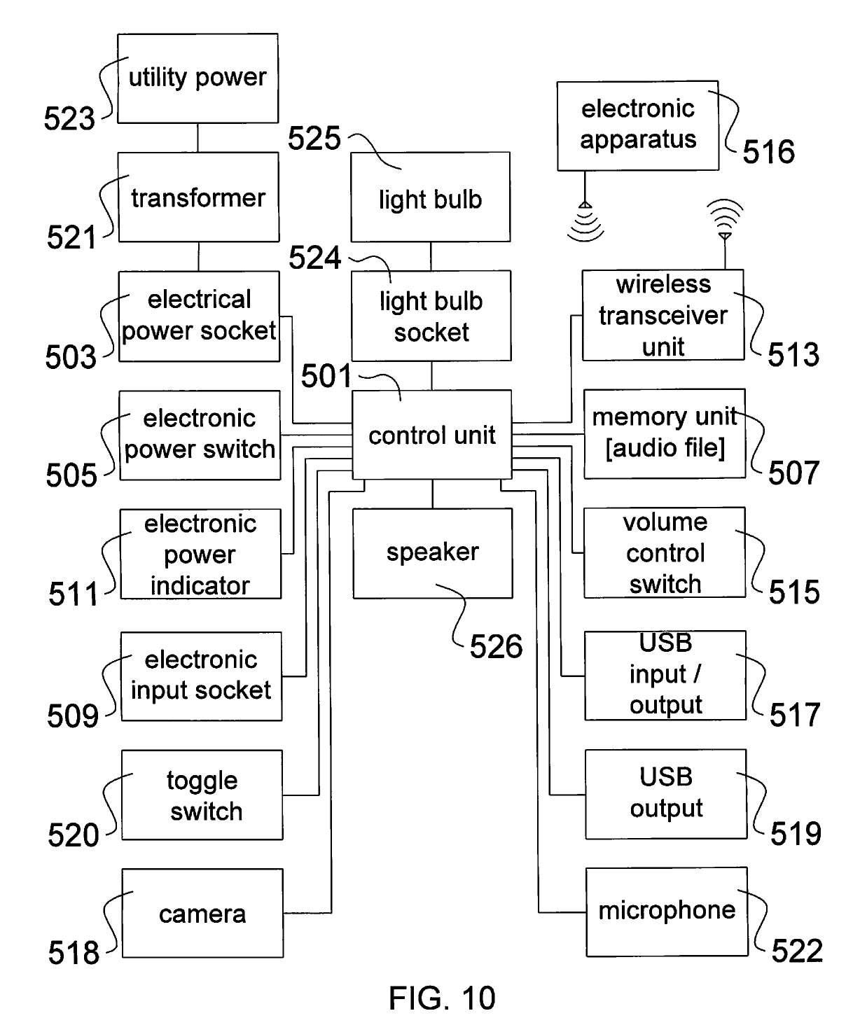

FIG. 10 illustrates a block diagram of the electronics and communications of the Himalayan salt rock system 100 including the Himalayan rock salt housing 101.

FIG. 10 illustrates a block diagram of the present invention and illustrates a control unit 501 being connected to an electrical power socket 503 to supply power, being connected to an electronic power switch 505 to control the on/off functions, being connected to a memory unit 507 which may store audio files for playback, being connected to electronic input socket 509, and being connected to an electronic power indicator 511 to provide a visual indication of the power.

The control unit 501 may be connected to the wireless transceiver unit 513 which receives signals from the electronic apparatus 515, may be connected to the volume control switch 515 to control the volume of the speaker 14, may be connected to the electronic output socket 517 for control of an electronic device (not shown) and may be connected to another electronic output socket 519 for control of another electronic device (not shown). A transformer 521 may be connected to the electrical power socket 503 and may be connected to the utility power 523.

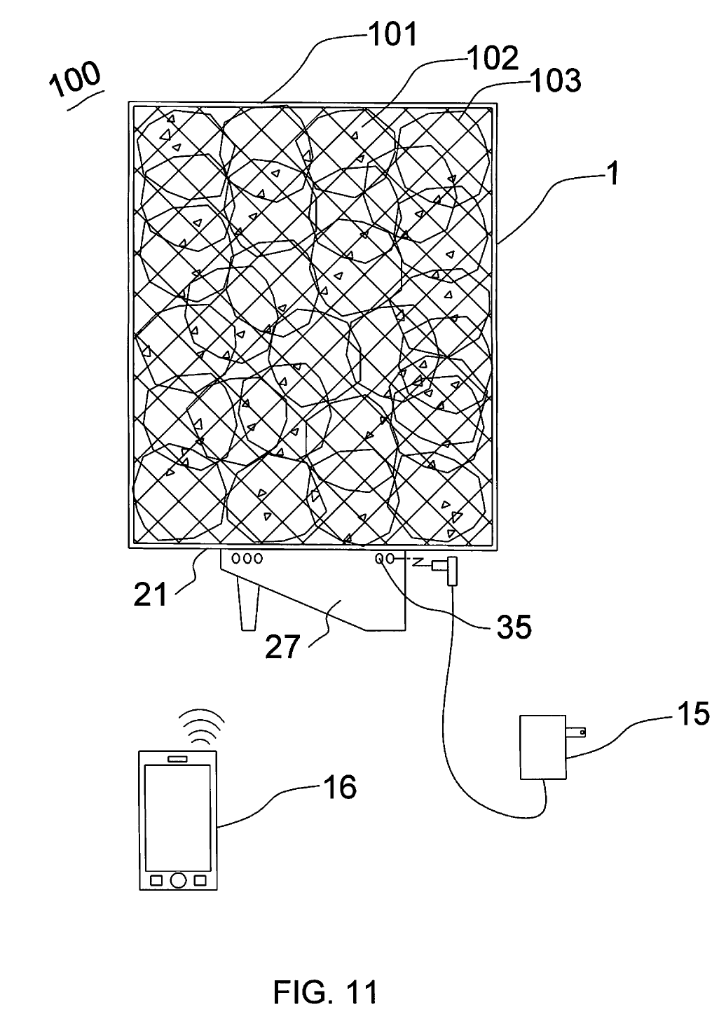

FIG. 11 illustrates a view of the another embodiment of the Himalayan salt rock system. FIG. 11 illustrates the Himalayan salt rock system 100 being powered by a power supply 15 which may include a transformer to reduce the voltage supplied to the base 27 and illustrates a remote controller 16 controlling the Himalayan salt rock system 100 wirelessly and remotely.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A speaker system to distribute sound, comprising:
a housing formed from only Himalayan salt rock having a central aperture;
a platform being positioned within the central aperture;
a light source being mounted on the platform;
a base connected to the housing;
a speaker connected to the base at an acute angle.

2) A speaker system to distribute sound as in claim 1, wherein the light source is an incandescent type of bulb.

3) A speaker system to distribute sound as in claim 1, wherein the light source is a multi-spectrum LED light.

4) A speaker system to distribute sound as in claim 1, wherein the speaker is a Bluetooth speaker.

5) A speaker system to distribute sound as in claim 1, wherein the speaker is a Wi-Fi speaker.

6) A speaker system to distribute sound as in claim 1 wherein the base includes a toggle switch to receive different sound sources.

7) A speaker system to distribute sound as in claim 1, wherein the base includes a power indicator.

8) A speaker system to distribute sound as in claim 1, wherein the base includes a USB port.

9) A speaker system to distribute sound as in claim 1, wherein the base includes a far field microphone.

10) A speaker system to distribute sound as in claim 1, wherein the base includes a volume control for the speaker.

11) A speaker system to distribute sound as in claim 1, wherein the base includes an audio jack.

12) A speaker system to distribute sound as in claim 1, wherein the base includes a camera.
